(19) United States
(12) Patent Application Publication
(10) Pub. No.: US 2024/0401132 A1
(43) Pub. Date: Dec. 5, 2024

(54) Rolling Circle Amplification-Coupled Glass Nanopore Counting of Mild Traumatic Brain Injury-Related Salivary miRNAs

(71) Applicant: THE PENN STATE RESEARCH FOUNDATION, University Park, PA (US)

(72) Inventors: Weihua Guan, State College, PA (US); Ming Dong, State College, PA (US); Zifan Tang, San Diego, CA (US)

(21) Appl. No.: 18/693,441

(22) PCT Filed: Sep. 20, 2022
(86) PCT No.: PCT/US2022/044089

§ 371 Date: Mar. 19, 2024

Related U.S. Application Data
(60) Provisional application No. 63/246,851, filed on Sep. 22, 2021.

(51) Int. Cl. C12Q 1/6876; C12Q 1/682

(52) U.S. Cl. CPC C12Q 1/6876; C12Q 1/682; C12Q 2600/118; C12Q 2600/178

(57) ABSTRACT

A method of counting target salivary miRNAs related to mild traumatic brain injury (mTBI) includes the steps of binding the miRNAs to padlock probes specific to each miRNA forming a hybridized complex for each miRNA, ligating the hybridized complex forming a closed circular structure, elongating the hybridized complex producing an elongated ssDNA amplicon via rolling circle amplification (RCA) elongation, measuring the concentration of elongated ssDNA amplicon according to a translocation event rate using a solid-state pore structure with a diameter greater than 10 nm, and determining initial concentrations of miRNAs based on the quantity of the initial miRNA molecule which is linear with the concentration of elongated ssDNA amplicon.

### REFERENCE TO RELATED APPLICATION

This application is the U.S. National Stage of PCT/US2022/044089 filed on Sep. 20, 2022, which claims priority from U.S. Provisional Patent Application Ser. No. 63/246,851, filed Sep. 22, 2021, the entire content of which is incorporated herein by reference.

### FIELD OF THE INVENTION

This invention relates generally to counting of miRNAs and, in particular, to counting of mild traumatic brain injury-related salivary miRNAs using rolling circle amplification-coupled glass nanopore.

### BACKGROUND OF THE INVENTION

Mild traumatic brain injury (mTBI), or concussion, is the most common type of traumatic brain injury¹. The mTBI symptoms include headaches, fatigue, depression, anxiety and irritability, as well as impaired cognitive function. Yet, it is well known that mTBI is both underdiagnosed and underreported due to delayed onset of symptoms and the conventional subjective assessment methods like cognitive testing and symptom scale¹. Objective, rapid and accurate mTBI diagnosis remains as an unmet need for effectively managing the mTBI. Several technologies for objective mTBI diagnosis have been proposed, including neuroimaging², electrophysiology³, and blood biomarkers⁴. However, these existing technologies were not without challenges. For example, while changes of proteins and lipids in the blood were used to determine the risk of intracranial bleeding, most mTBIs do not result in intracranial bleeding⁵. Besides, those blood biomarkers are typically present at low concentrations (fM to pM), susceptible to degradation, and may have difficulty crossing the blood-brain barrier in cases of mTBIs⁶. On the other hand, neuroimaging and electrophysiology require expensive equipment and specialist interpretation². The long turnaround time and complex workflow of these existing technology preclude their adoption for rapid diagnosis of the mTBI, particularly at the point-of-care testing.

Recent findings suggested that salivary miRNAs are promising biomarkers for mTBI diagnosis based on their varied expression levels⁷. miRNAs are small single-stranded non-coding molecules that function in RNA silencing and post-transcriptional regulation of gene expression⁸. Since the saliva can be obtained non-invasively, salivary miRNA represents an ideal biomarker for rapid mTBIs diagnosis. However, detecting and differentiating miRNAs are challenging due to their short length and high homogeneity⁹. The common techniques for miRNA profiling include northern blotting¹⁰, RT-PCR¹¹, microarrays¹², and next-generation sequencing (NGS)¹³. While readily available and effective, these methods fall short of the requirement for rapid, inexpensive and accurate miRNA profiling for mTBI diagnosis. For instance, northern blotting has a complex workflow and requires radioactive label¹⁴. The primer efficacy in RT-PCR and the hybridization in microarrays is limited by the short length of miRNA. The turnaround time and the cost of NGS are still prohibitive for routine clinical adoption¹³. To the end of rapid and accessible mTBI diagnosis using salivary miRNAs, alternative approaches have been investigated, such as nanoparticle-derived probes¹⁵, electrochemical methods¹⁶ and isothermal amplification¹⁷. Among them, rolling circle amplification (RCA) is one of the isothermal methods to detect miRNAs with relatively short turnaround time and simple workflow. Due to the specificity required by the hybridization and ligation process, even one nucleotide difference in miRNA can be discriminated via RCA assay¹⁸.

### SUMMARY OF THE PRESENT INVENTION

Mild traumatic brain injury (mTBI) could be underdiagnosed and underreported due to the delayed onset of symptoms and the conventional subjective assessment. Recent studies suggested that salivary microRNAs (miRNAs) could be reliable biomarkers for objective mTBI diagnosis.

The present invention provides a platform and a method of counting of miRNAs using solid-state pore structures in combination with the rolling circle amplification process. In particular, the present method provides a method of counting of mild traumatic brain injury-related salivary miRNAs using rolling circle amplification (RCA)-coupled resistive pulse counting platform for profiling mTBI-related miRNAs, using easy-to-fabricate large solid-state pore structures. The method relies on the linear and specific elongation of the miRNA to a much larger RCA product, which the large solid-state pore structure can digitally count with a high signal-to-noise ratio.

Salivary miRNAs are promising biomarkers for mTBI diagnosis based on their varied expression levels. The present method provides a method for rapid and accurate mTBI diagnosis based on counting of salivary miRNAs using RCA-coupled pore structure. The pore structure might be a micropore, sub-micron pore or a nanopore.

The pore structure might be made of glass or other materials including but not limited to Si, SiO2, SiNx, ZrO2, HfO2, TiO2 (oxide dielectric material), 2D materials such as hexagonal boron nitride (h-BN) and Transition Metal Dichalcogenides (MoS2, WS2, MoSe2), or polymer materials such as PDMS.

Padlock probes can be designed to specifically target the miRNAs. The miRNA will first bind to a probe specific to the miRNA forming a hybridized complex. The hybridized complex will be further ligated to form a closed circular structure. Then the hybridized miRNA is elongated using the probe as a template through the RCA elongation process. The RCA elongation process will produce an amplicon, i.e., a long ssDNA product which can be easily detected by the glass sub-micron pore with a high signal-to-noise ratio due to its large size of the ssDNA product. The elongated ssDNA product might be greater than 70 k nucleotides.

Due to the specificity required by the hybridization and ligation process, even one nucleotide difference in miRNA can be discriminated via the RCA assay. A linear relationship is observed between the measured event rate and the initial quantity of miRNAs such that the pore event rate of the amplicons is an excellent measurement of the initial miRNA concentrations.

Specificity also enables multiplexed miRNA profiling, meaning that parallel testing can be effectively run for multiple miRNA targets and each testing corresponds to a single specific miRNA target. The analyte sample will be aliquoted into separate reactions and each of these reactions has a specific probe and the pore structure detector. The pore structures read these reactions in parallel.

A large pore is used to avoid signals generated by small molecules like miRNAs and padlock probes. Small molecules like miRNAs and probes can not be detected by the sub-micron pore.

A large pore is not imagined for analyzing miRNAs due to the clear mismatch of the size. Often the “resistive pulse sensing” technique would require the orifice size no more than 5 times of the analyte size. In some embodiments of the present invention, the pore of a few 100s nanometers to a few um is about 100-1000 times the miRNA size. The diameter of the micropore may range from 10 nm-5 μm, or preferably at 100-500 nm, or even more preferably at 200-300 nm, or preferably at 250 nm.

Examples of the target miRNAs biomarkers for mTBI might be let-7a, miR-30e or miR-21.

### BRIEF DESCRIPTION OF THE DRAWINGS

FIG. 1A shows a schematics of the RCA-based miRNA detection method; (7aP, 30eP and 21P denote the padlock probe for let-7a, miR-30e and miR-21, respectively. The red region of 7aP, blue region of 30eP and green region of 21P is complementary to let-7a, miR-30e and miR-21, respectively);

FIG. 1B shows a gel image of RCA products;

FIG. 1C shows representative current traces for RCA reactions with probes only and for RCA reactions with both padlock probes and the target miRNA;

FIG. 1D shows distribution of dwell time and peak current for blockage events;

FIG. 1E shows a graph of the measured event rate as a function of the input miRNA concentration;

FIG. 2 shows a table of miRNAs and padlock probes sequences used in the examples of the present invention;

FIG. 3A shows a schematics of RCA-coupled nanopore counting setup; (The enlarged SEM image shows a typical glass nanopore (˜200 nm in diameter) used in our experiments. We intentionally used this large pore to avoid signals generated by small molecules like miRNAs and padlock probes);

FIG. 3B shows IV characterization of the glass nanopore; (The conductance is about 370nS);

FIG. 3C shows the current trace of the glass nanopore tested in Tris-EDTA buffered 1 M KCl;

FIG. 3D shows the power spectral density (PSD) of the current trace in FIG. 3C;

FIG. 4A shows current traces for the 7aP probe-only reactions for background false positive rate evaluation; (For 7aP probe-only reaction, 5 events captured within 10 mins (i.e., <0.008 s⁻¹ false positive rate). Orange triangles annotate the captured events);

FIG. 4B shows current traces for the 30eP probe-only reactions for background false positive rate evaluation; (For 30eP probe-only reaction, 5 events captured within 10 mins (i.e., <0.008 s⁻¹ false positive rate));

FIG. 4C shows current traces for the 21P probe-only reactions for background false positive rate evaluation; (For 21P probe-only reaction, 3 events captured within 10 mins (i.e., <0.005 s⁻¹ false positive rate));

FIGS. 5A-5D show the typical RCA product translocation events for the let-7a panel;

FIG. 6 shows normalized distributions of interarrival time for different miRNAs with monoexponential fits; (The exponential distribution of the interarrival time between events indicates the translocation events follow a Poisson process, indicating the translocations are random and independent);

FIG. 7A shows 10 mins current trace of the 0 fmol let-7a RCA assay without total RNA background;

FIG. 7B shows 10 mins current trace of the 0 fmol let-7a RCA assay with total RNA background;

FIG. 7C shows 10 mins current trace of the extracted salivary total RNA without RCA assay;

FIG. 7D shows gel electrophoresis of extracted salivary total RNA; (Most of the RNAs have a length shorter than 500 nucleotides);

FIG. 8A shows the gel image of the RCA products with different quantities of the purified let-7a (without human salivary total RNA);

FIG. 8B shows the corresponding current traces obtained in nanopore sensing;

FIG. 8C shows extracted event rate as a function of the let-7a quantity;

FIG. 8D shows the gel image of the RCA products with different let-7a quantities in the salivary total RNA background;

FIG. 8E shows the corresponding current traces obtained in nanopore sensing with salivary total RNA background;

FIG. 8F shows the extracted nanopore event rate as a function of the let-7a quantity with salivary total RNA background;

FIG. 9A shows the gel image of the RCA products with different quantities of the purified miR-30e (without human salivary total RNA);

FIG. 9B shows the corresponding current traces obtained in nanopore sensing;

FIG. 9C shows the extracted event rate as a function of the miR-30e quantity;

FIG. 9D shows the gel image of the RCA products with different miR-30e quantities in the salivary total RNA background;

FIG. 9E shows the corresponding current traces obtained in nanopore sensing with salivary total RNA background;

FIG. 9F shows the extracted nanopore event rate as a function of the miR-30e quantity with salivary total RNA background;

FIG. 10A shows the gel image of the RCA products with different quantities of the purified

miR-21 (without human salivary total RNA);

FIG. 10B shows the corresponding current traces obtained in nanopore sensing;

FIG. 10C shows the extracted event rate as a function of the miR-21 quantity;

FIG. 10D shows the gel image of the RCA products with different miR-21 quantities in the salivary total RNA background;

FIG. 10E shows the corresponding current traces obtained in nanopore sensing with salivary total RNA background;

FIG. 10F shows the extracted nanopore event rate as a function of the miR-21 quantity with salivary total RNA background;

FIG. 11A shows the gel image of RCA products for different combinations of miRNAs and padlock probes. Each RCA reaction was performed with 160 fmol probes and 40 fmol miRNAs;

FIG. 11B shows the corresponding current traces for each miRNA and padlock combination. Evident events were only visible in the specific combinations;

FIG. 12A shows let-7a RCA assay added with 30eP probe;

FIG. 12B shows let-7a RCA assay with 21P probe added;

FIG. 12C shows miR-30e RCA assay with 7aP probe added;

FIG. 12D shows miR-30e RCA assay with 21P probe added;

FIG. 12E shows miR-21 RCA assay with 7aP probe added;

FIG. 12F shows miR-21 RCA assay with 30eP probe added. The false positive rates of all the non-specific combinations were smaller than 0.003 s⁻¹;

FIG. 13A shows the gel image of the RCA products for three mixed samples with varying quantities of let-7a, miR-30e and miR-21. Sample 1 contains 20 fmol let-7a, 40 fmol miR-30e and 80 fmol miR-21; Sample 2 contains 40 fmol let-7a, 40 fmol miR-30e and 40 fmol miR-21; Sample 3 contains 80 fmol let-7a, 40 fmol miR-30e and 20 fmol miR-21. Each of these mock samples was parallelly reacted with a specific padlock probe;

FIG. 13B shows the measured event rates for each of the three mixed samples; and

FIG. 13C shows the measured individual miRNA concentration versus the input miRNA concentration for each of three mixed samples. The solid line denotes the expected value. The error bars represent the Poisson uncertainty.

### DETAILED DESCRIPTION OF THE PRESENT INVENTION

### Principle of the Present Invention

The embodiments of the present invention provide a method of counting of miRNAs using a solid-state pore structure in combination with the rolling circle amplification process. In particular, the present method provides a method of counting of mild traumatic brain injury-related salivary miRNAs using rolling circle amplification (RCA)-coupled pore structure.

Recent studies have shown that miRNAs expression levels could be up-regulated after mTBI. For example, previous studies have shown that mTBI-related miRNAs could increase two times for positive patients²¹⁻²⁴. Salivary miRNAs are promising biomarkers for mTBI diagnosis based on their varied expression levels. The present method provides a method for rapid and accurate mTBI diagnosis based on counting of salivary miRNAs using RCA-coupled pore. The pore might be a micropore, sub-micron pore or a nanopore.

The pore structure might be a glass pore or made from other materials including but not limited to Si, SiO2, SiNx, ZrO2, HfO2, TiO2 (oxide dielectric material), 2D materials such as hexagonal boron nitride (h-BN) and Transition Metal Dichalcogenides (MoS2, WS2, MoSe2), or polymer materials such as PDMS.

Padlock probes can be designed to specifically target the miRNAs. The miRNA will first bind to its specific probe forming a hybridized complex. The hybridized complex will be further ligated to form a closed circular structure. Then the hybridized miRNA is elongated using the probe as a template through the RCA elongation process. The RCA elongation process will produce an amplicon, i.e., a long ssDNA product which can be easily detected by the glass sub-micron pore with a high signal-to-noise ratio due to its large size of the ssDNA product. Due to the specificity required by the hybridization and ligation process, even one nucleotide difference in miRNA can be discriminated via the RCA assay. A linear relationship is observed between the measured event rate and the initial quantity of miRNAs such that the pore event rate of the amplicons is an excellent measurement of the initial miRNA concentrations.

A large pore is used to avoid signals generated by small molecules like miRNAs and padlock probes. Small molecules like miRNAs and probes can not be detected by the sub-micron pore.

A micropore is not imagined for analyzing miRNAs due to the clear mismatch of the size. Often the “resistive pulse sensing” technique would require the orifice size no more than 5 times of the analyte size. In some embodiment, the pore of a few 100s nanometers to a few um is about 100-1000 times the miRNA size. These large pores are cost-effective, repeatable, and robust to manufacture. The diameter of the micropore may range from 10 nm-5 um, or preferably at 100-500 nm, or even more preferably at 200-300 nm, or preferably at 250 nm.

The present invention provides a scalable multiplexed miRNA analysis apparatus enabled by manufacturable pores larger than 10 nm.

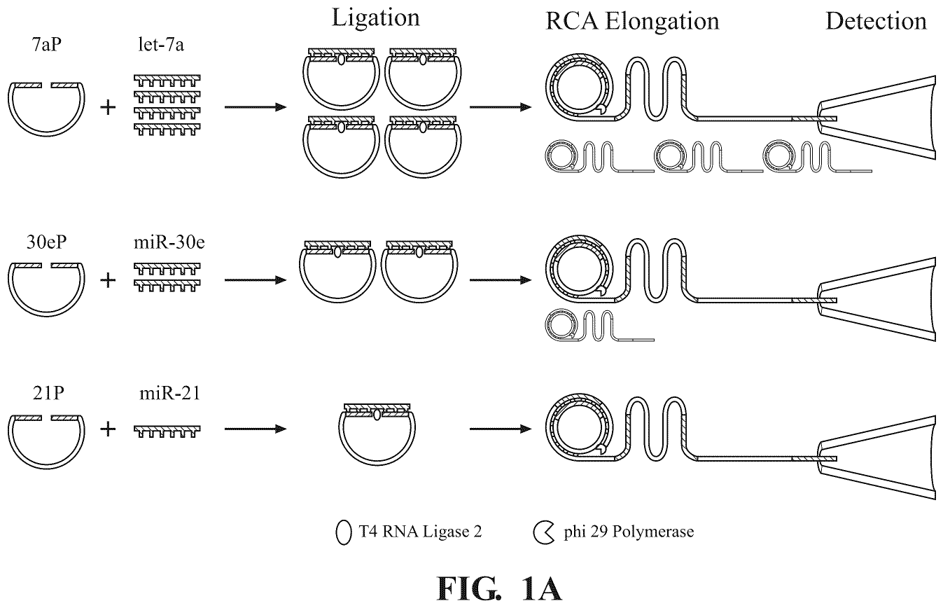

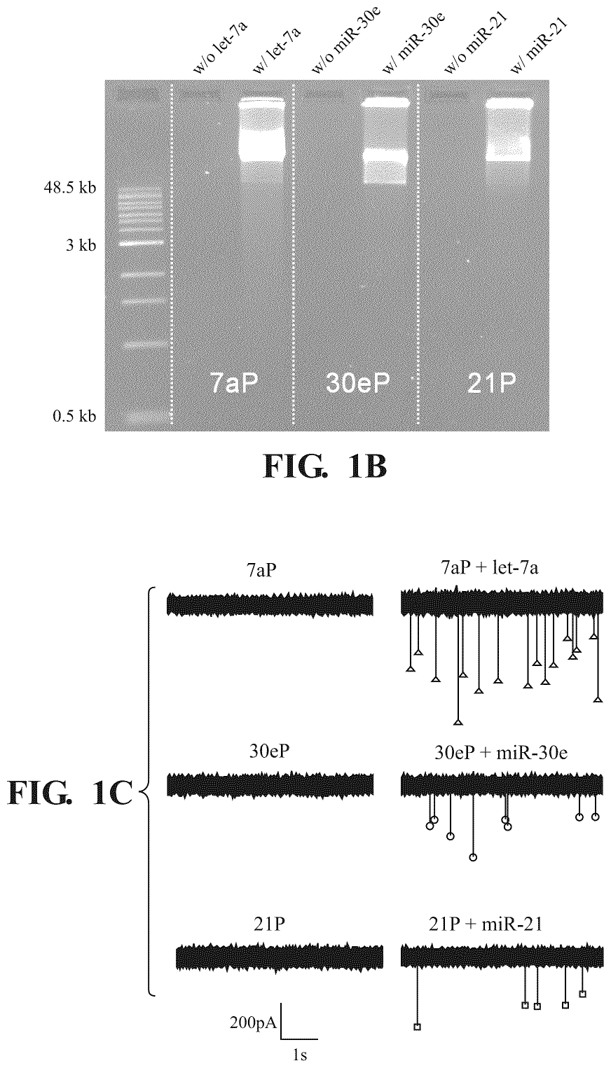

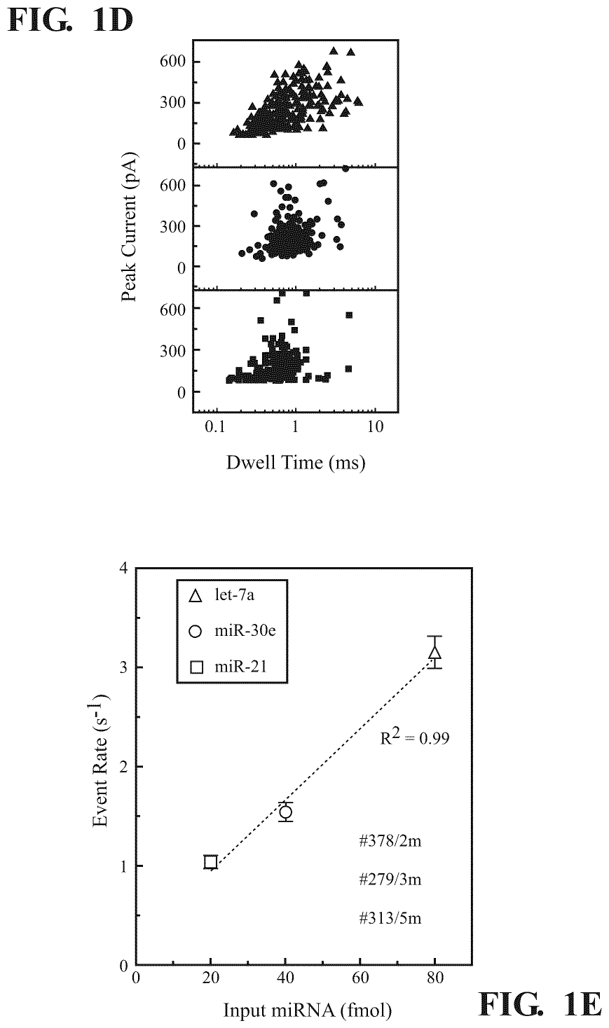

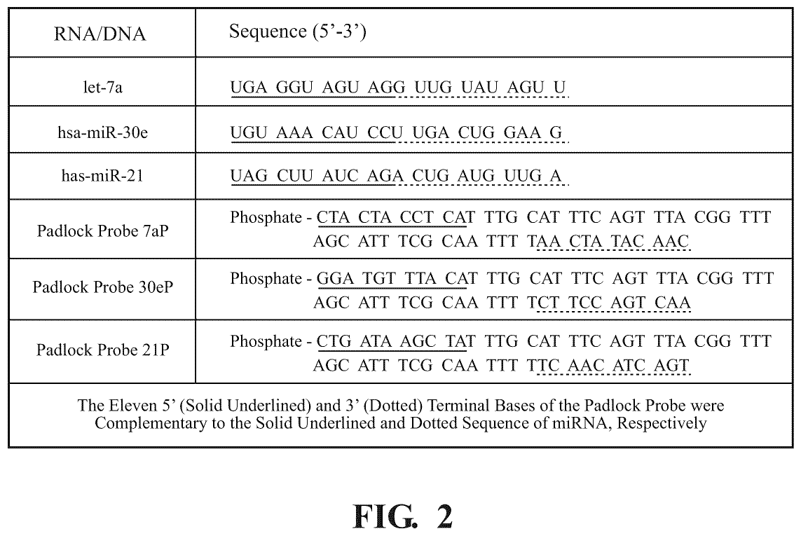

FIG. 1A shows the principle of the RCA-coupled glass nanopore counting of miRNAs. A subset of panels were chosen: let-7a (65% increased)²¹, miR-30e (88% increased)²², and miR-21 (280% increased)^{23,24}. Padlock probes 18 were designed to specifically target the let-7a, miR-30e, and miR-21 (see the table of FIG. 2 for detailed probe design). As shown in FIG. 1A, the miRNA will first bind to its specific probe. The hybridized complex will be further ligated by the T4 RNA ligase 2 to form a closed circular structure. After that, the phi29 DNA polymerase is introduced to elongate the hybridized miRNA using the probe as a template (RCA elongation). The RCA elongation process will produce a long ssDNA product greater than 70 k nucleotides²⁵. This ssDNA product can be easily detected by the glass sub-micron pore with a high signal-to-noise ratio due to its large size. In contrast, small molecules like miRNAs and probes can not be detected. The event rate of products will be counted through the nanopore without sizing by event shape. This is due to the RCA products themselves could have a size distribution, and products could conform during translocation. By measuring the concentration of the enlarged ssDNA product through the event rate²⁶, one can determine the initial miRNA concentrations since the quantity of the initial miRNA molecule is linear with the number of elongated ssDNA products.

### Experimental Setup

### Materials and Chemicals

RNAs and DNAs were synthesized by Integrated DNA Technologies (IDT), the detailed sequences are listed in the table of FIG. 2. Nuclease-free molecular biology grade water was from NEB (B1500S). DNA gel blue loading dye (6×, B7021S) was from NEB. Agarose was from Fisher Scientific (BP160100). DNA ladder was from NEB (N3239S). SYBR Gold nucleic acid gel stain (S11494) was from NEB. Deoxynucleotide solution mix, T4 RNA ligase 2 and Phi29 DNA polymerase were purchased from NEB. The salivary total RNA was extracted using ChargeSwitch Total RNA Cell Kit from Invitrogen. Ag/AgCl electrodes were house-made with 0.375 mm Ag wires (Warner Instruments, Hamden, USA). Potassium chloride and 1× Tris-EDTA buffer solution (10 mM Tris-HCl, 1 mM disodium EDTA, pH 8.0) were purchased from Sigma-Aldrich. The solution was filtered with a 0.2 μm Anotop filter (Whatman) and degassed in a vacuum chamber prior to use.

### 2. Rolling Circle Amplification Assay

For the ligation reactions, the reaction mixture consisted of nuclease-free water, ligation buffer (50 mM Tris-HCl, pH 7.5, 2 mM MgCl₂, 10 mM dithiothreitol (DTT), 400 mM ATP), 2 U of T4 RNA ligase 2, 160 fmol padlock probes unless otherwise stated, and the target miRNAs (single miRNA or miRNA mixtures) in a reaction volume of 10 μL. Before the ligase and ligation buffer were added, the reaction mixture was heated at 55° C. for 5 mins and annealed to 39° C. at −1° C./min in the C1000 Touch Thermal Cycler (Bio-Rad, USA). The ligase and the buffer were then added and the reaction mixture was incubated at 39° C. for 45 mins. The products of the ligation reaction were added to the 10 μL RCA reaction mixture containing 80 mM Tris-HCl (pH 7.5), 100 mM KCl, 20 mM MgCl₂, 10 mM (NH₄)₂SO₄, 8 mM DTT, 500 mM of each dNTP, and 20 U of phi29 DNA polymerase. The RCA reactions were performed at 30° C. for 30 mins.

### 3. Gel Analysis

The reaction mixture for gel electrophoresis was terminated by adding 4 μL gel blue loading dye, and the 1.0% agarose gel (made with 1×TBE buffer) was running for 1 h at 120 V. After that, SYBR Gold nucleic acid gel stain was used to stain the gel for 30 mins. Gel electrophoresis images were acquired with a GelDoc Go imaging system (Bio-Rad, USA).

### 4. Salivary Total RNA Extraction

The saliva samples were collected from healthy volunteers. The total RNA was extracted from 1 mL saliva by ChargeSwitch™ total RNA cell kit following the protocol. The isolated total RNA was eluted with 75 μL elution buffer. The final concentration of extracted RNA was measured by Nanodrop 2000 (Thermo Fisher Scientific) as 9.6 ng/μL. The synthetic let-7a was spiked into 7 μL extracted saliva RNA solution at various quantities ranging from 10 to 160 fmol.

### 5. Glass Nanopore Fabrication

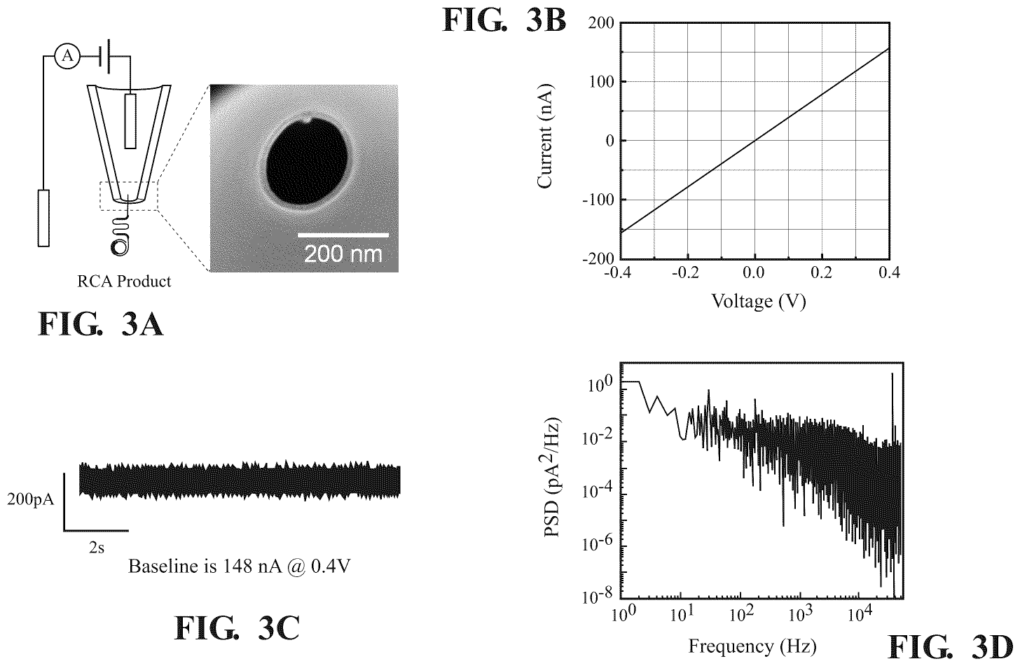

The quartz capillaries (QF120-90-7.5; Sutter Instrument Co, USA.) were cleaned by piranha for 30 mins to remove organic contaminants, then rinsed with DI water and dried in the oven at 100° C. for 30 mins. The capillaries were oxygen plasma cleaned for 5 mins to enhance the hydrophilic property. The capillary was then pulled by a laser pipet puller (P-2000, Sutter Instruments, USA) using a two-line program: (1) Heat 575, Filament 3, Velocity 35, Delay 145,and Pull 75; (2) Heat 425, Filament 0, Velocity 15, Delay 128, and Pull 185. This recipe typically produced pores with a diameter of 217±9 nm. The SEM image and electrical properties of a typical pore are shown in FIG. 3A. Due to the influence of humidity and temperature, the pulling parameters should be modified accordingly. After pulling, the capillary was filled with Tris-EDTA buffered 1M KCl solution immediately using a micro-injector.

### 6. Nanopore Sensing and Data Analysis

The 20 μL RCA reaction mixture for nanopore sensing was terminated by adding 80 μL Tris-EDTA buffered 1.25 M KCl solution to form 100 μL of testing sample. The IM KCI filled glass pore was fixed by a pipette holder and immersed in the PCR tube containing the 100 μL testing sample. Ag/AgCl electrodes were placed inside the glass capillary as well as in the test sample solution. A typical voltage of 400 mV was applied across the pore by 6363 DAQ card (National Instruments, USA). A trans-impedance amplifier (Axopatch 200B, Molecular Device, USA) was used to amplify the resulting current and then digitized by the 6363 DAQ card at 100kHz sampling rate. Finally, a customized MATLAB (MathWorks) software was used to analyze the current time trace and extract the single molecule translocation information. The threshold of event peak was set at 5 times of standard deviation of the current traces. If clogging was observed, five times IV sweeps from −500 mV to 500 mV were applied to restore the pore.

### Validation

Prior to the glass nanopore quantification experiment, the RCA assay for let-7a, miR-30e and miR-21 was validated. As shown in the gel results in FIG. 1B, reactions without the miRNA input (i.e., with probes only) produced no elongated product, whereas reactions with the miRNAs showed the product with a length much larger than 48.5 kb. This confirmed that let-7a, miR-30e and miR-21 can be successfully elongated to their corresponding ssDNA products through the RCA reaction.

After confirming there was indeed ssDNA amplicons been produced, these amplicon solutions are tested with the glass sub-micron pore sensor. In an example, a glass pore used in our experiment is about 200 nm in diameter. FIG. 3A shows the schematics of RCA-coupled nanopore counting setup. The enlarged SEM image shows a typical glass nanopore (˜200 nm in diameter). This large pore is used to avoid signals generated by small molecules like miRNAs and padlock probes. FIG. 3B shows IV characterization of the glass nanopore. The conductance is about 370 nS. We applied a voltage of 400 m V across the pore and counted the translocation events by monitoring the ionic current. FIG. 3C shows the current trace of the large glass nanopore tested in Tris-EDTA buffered 1 M KCl. FIG. 3D shows the power spectral density (PSD) of the current trace in FIG. 3C.

The nanopore counting was conducted until at least 250 events were captured to reduce the event rate uncertainty (≤6%)²⁶ or 10 mins were reached. As shown in the time traces in FIG. 1C, for the probe-only reactions (three left traces), there were no events observed during the 5 s of the measurement.

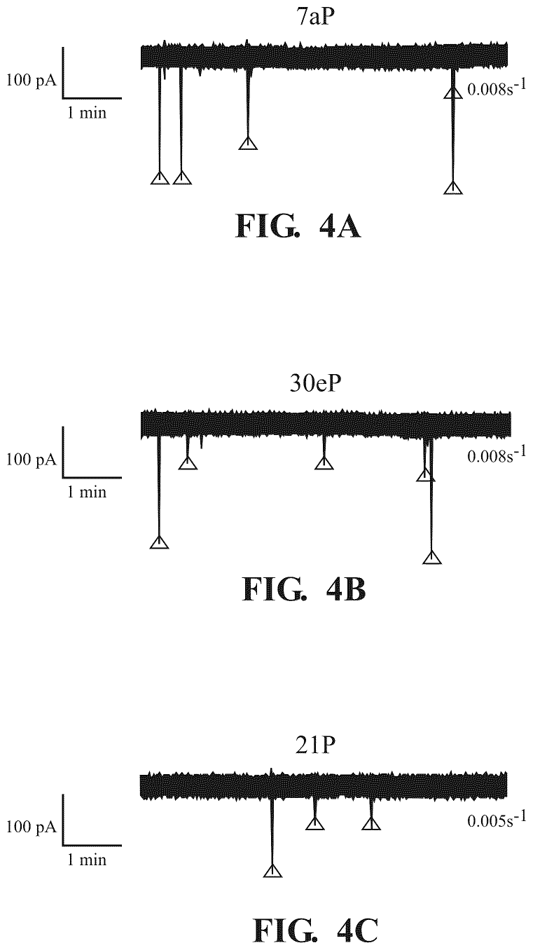

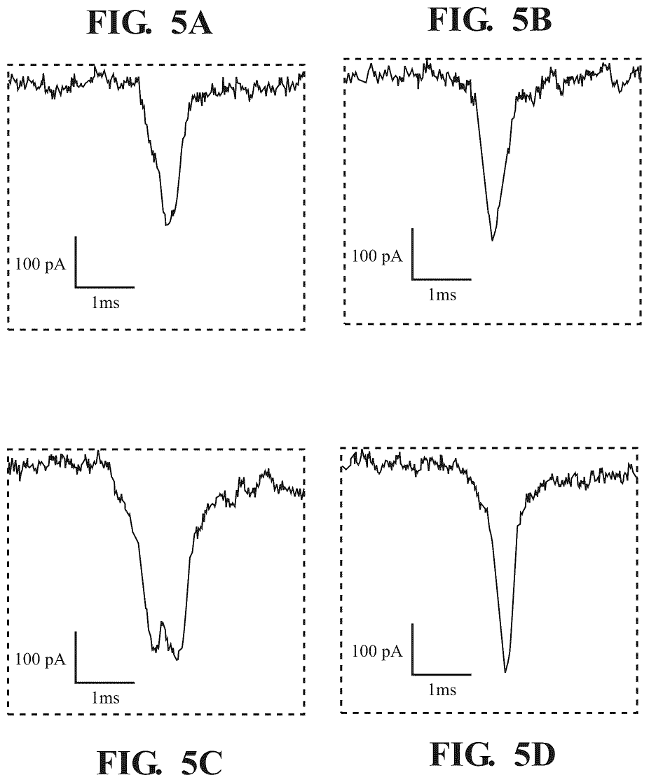

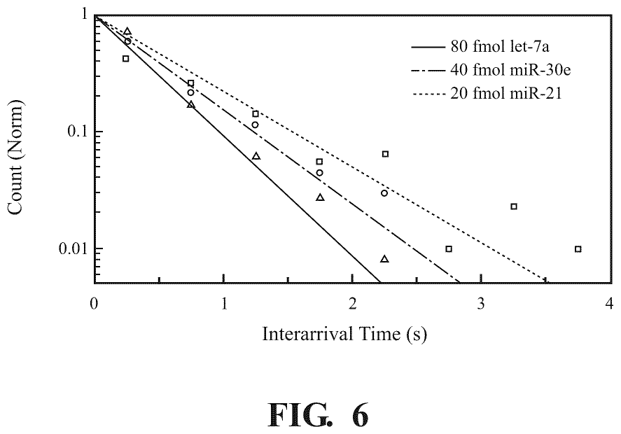

FIGS. 4A-4C show current traces for the probe-only reactions for background false positive rate evaluation. For 7aP probe-only reaction, as shown in FIG. 4A, 5 events were captured within 10 mins (i.e., <0.008 s⁻¹ false positive rate). Triangles annotate the captured events. For 30eP probe-only reaction, as shown in FIG. 4B, 5 events captured within 10 mins (i.e., <0.008 s⁻¹ false positive rate). For 21P probe-only reaction, as shown in FIG. 4C, 3 events captured within 10 mins (i.e., <0.005 s⁻¹ false positive rate). In fact, for a longer measurement of 10 mins, less than 5 events could be observed, indicating the background event rate was less than 0.008 s⁻¹. This negligible background event rate means that the padlock probes themselves cannot be detected by the pore due to their small size. In contrast, for the positive reactions (three right traces in FIG. 1C), clear blockage events were observed. FIGS. 5A-5D show representative single translocation event profiles. The exponential distribution of the interarrival time between events, as shown in FIG. 6, indicates the translocation events follow a Poisson process, which means the translocations are random and independent²⁷. Further analysis of these events revealed that the elongated amplicons for let-7a, miR-30e, and miR-21 were similar in their size distribution since their dwell time and peak current are comparable, shown in FIG. 1D. This is consistent with the gel results shown in FIG. 1B. Given the similar size of starting ligated products for let-7a, miR-30e and miR-21 shown in FIG. 1A, we indeed expect the ssDNA amplicons to be comparable in size after the same duration of RCA elongation.

While the dwell time versus peak current distributions was comparable for let-7a, miR-30e and miR-21 product, it is also evident that their event rate differs from each other, as shown in FIG. 1E (“#378/2 m” means 378 events were observed in two minutes measurement and the error bars represent the Poisson counting uncertainty n^{1/2}/T). This is because we intentionally used different quantities of these three miRNAs. We used 80 fmol of let-7a, 40 fmol of miR-30e, 20fmol of miR-21, together with 160 fmol of their corresponding padlock probes for the RCA reactions. Please note that we reported the miRNA quantity instead of the concentration throughout this work to avoid the possible confusion caused by the varying volumes of RCA buffers and nanopore measurement buffers. To examine if the measured amplicon event rate is quantitatively correlated to the miRNA concentration, the event rate was extracted for each of these samples and plotted it against the initial miRNA concentration, which is shown in FIG. 1E. As shown, there is an excellent correlation between the miRNA concentration and the nanopore event rate (R²=0.99). This linear correlation suggested that inter-miRNA profiling is feasible by the RCA-coupled glass nanopore counting platform.

Quantification of miMRNAs With and Without Salivary RNA Background

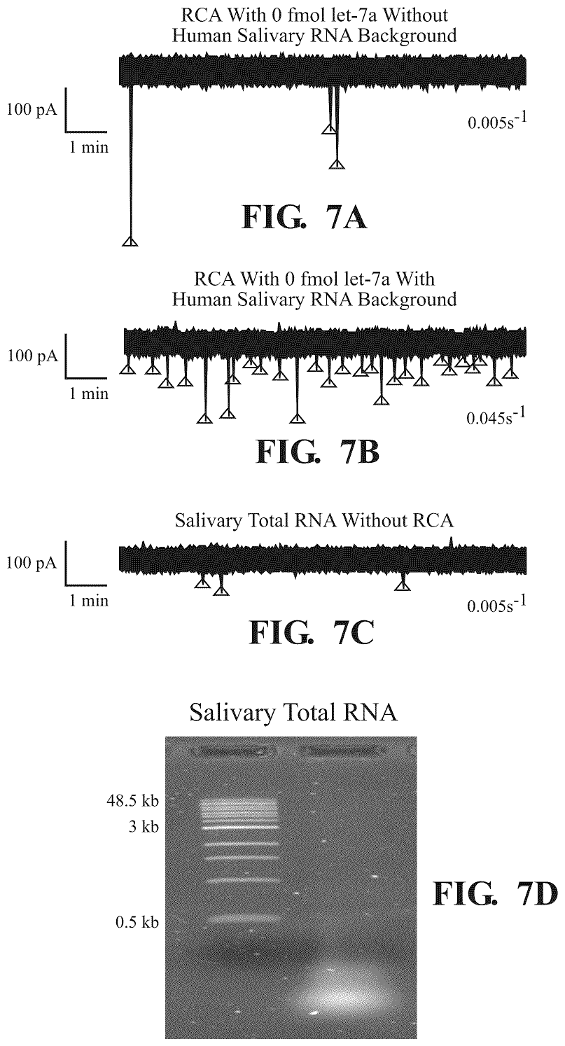

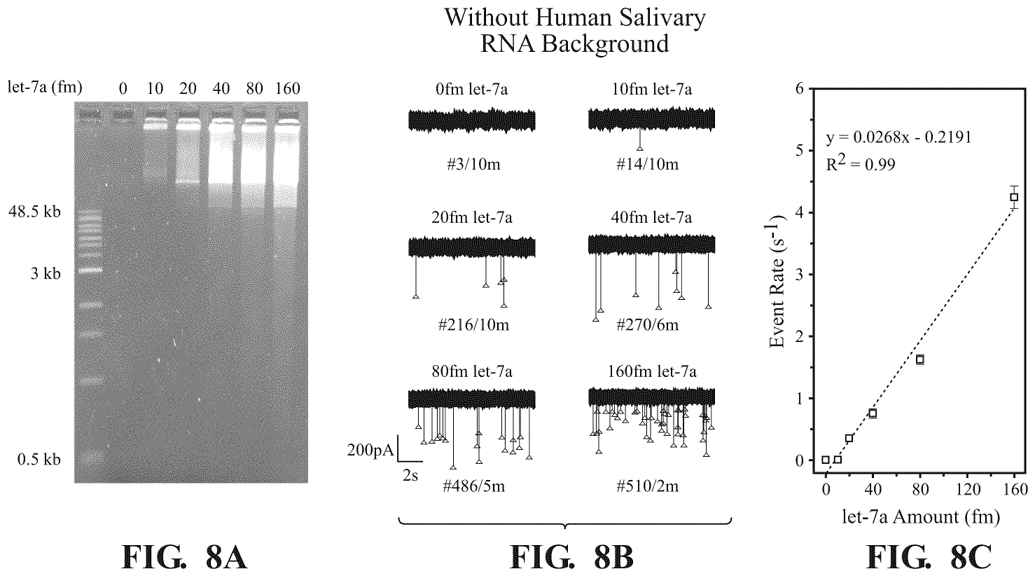

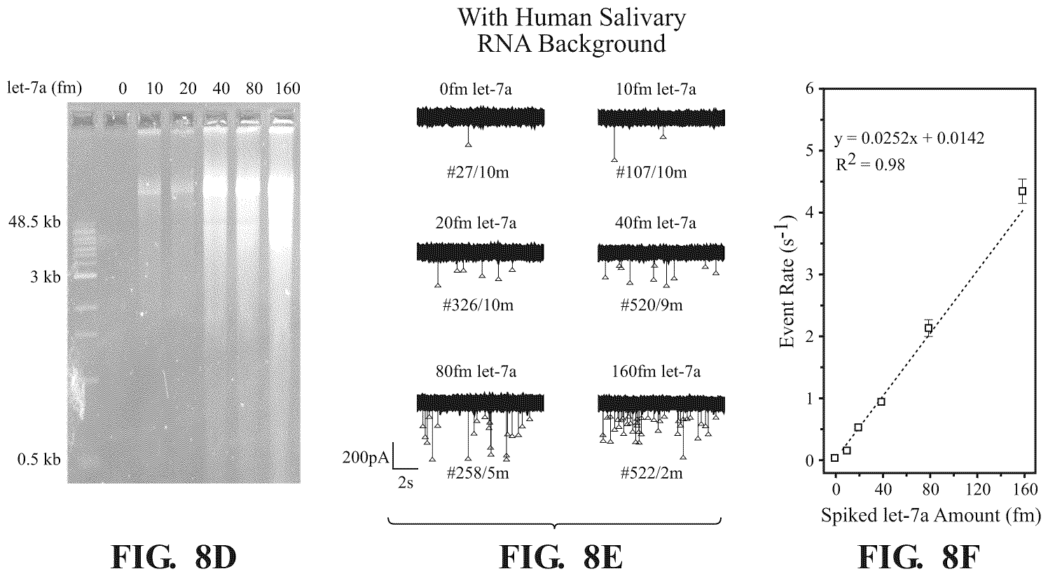

Previous studies have shown that mTBI-related miRNAs could increase two times for positive patients²¹⁻²⁴. To further evaluate the intra-miRNA quantification ability of the RCA-coupled nanopore counting platform, we prepared a 2× serial dilution of let-7a miRNAs and performed 30 mins of RCA reaction with let-7a quantities ranging from 0 to 160 fmol (corresponding to the clinically relevant miRNA concentration range of 0-160 pM²⁸ with 1 mL of raw saliva sample). The resulting RCA products were examined with gel (FIG. 8A). As shown, the RCA product concentration increases when the input let-7a miRNAs increases. This is not surprising as the padlock probes were excessively provided in all reactions. To quantify these RCA products, the nanopore counting experiment is performed. The representative 10s current traces at different let-7a quantities were shown in FIG. 8B. The background event rate observed for reactions without let-7a input was less than 0.005 s⁻¹, as shown in FIG. 7A. The event rate went from 0.023 s⁻¹ with 10 fmol let-7a to 4.250 s⁻¹ with 160 fmol let-7a, shown in FIG. 8B. FIG. 8C summarizes the correlation between the measured event rate and the initial let-7a quantity. A linear relationship with R² of 0.99 was observed, suggesting the nanopore event rate of the amplicons is an excellent measurement of the initial miRNA concentrations.

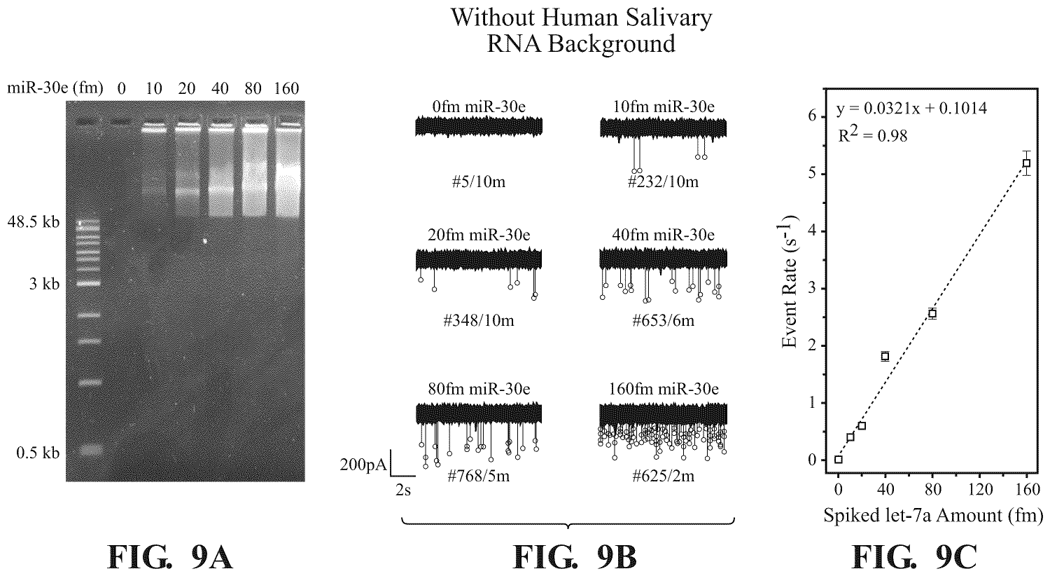

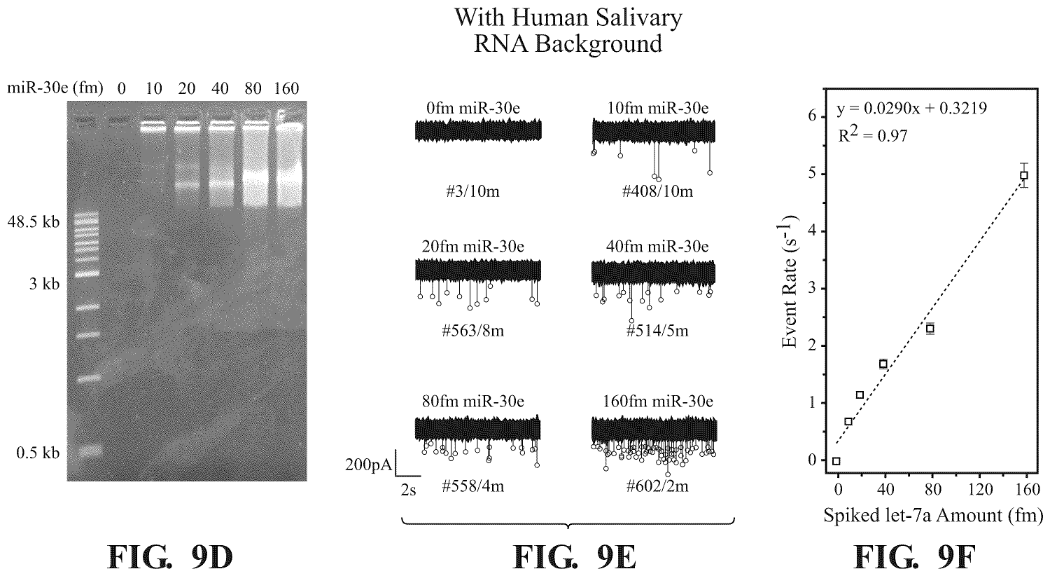

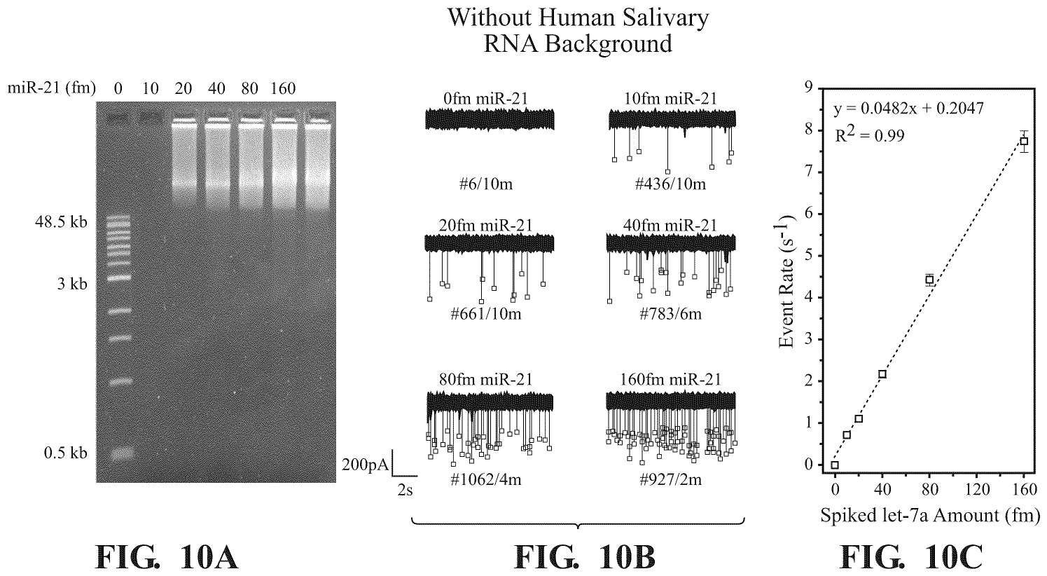

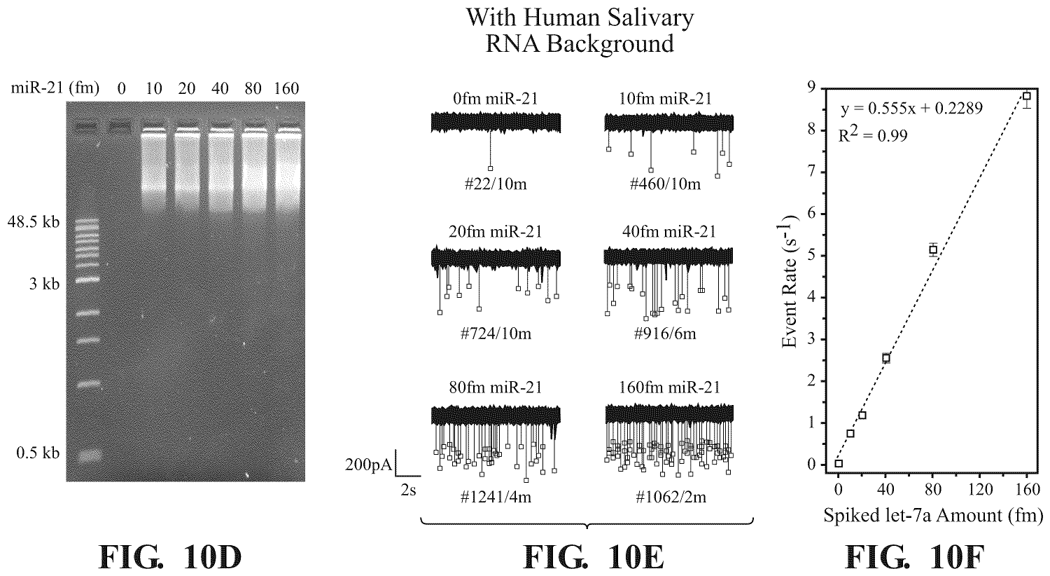

To further test if the salivary total RNA background would interfere with the nanopore counting, different concentrations of the purified let-7a were spiked into the salivary RNA background. We performed 30 mins of RCA elongation with these spiked samples, in which let-7a quantities range from 0 to 160 fmol and the padlock probe is 160 fmol. FIG. 8D presents the gel results from these reactions. Similar to the case without salivary RNA background, more input let-7a produced an increased amount of RCA amplicons with salivary RNA background. The nanopore counting was also performed on these RCA products. FIG. 8E presents the representative current traces. As expected, more translocation events were observed as more let-7a miRNAs were spiked. FIG. 8F plotted the event rate as a function of the initial let-7a quantity spiked into the salivary RNA background. As shown, there is also an excellent linear relationship with R² of 0.98. Interestingly, the event rate at each let-7a concentration is slightly higher with salivary RNA background than that without it. For example, the event rate observed for reactions of 0 fmol let-7a input was 0.045 s⁻¹ and 0.005 s⁻¹ with and without salivary RNA background, respectively, as indicated in FIGS. 7A and 7B. This increased background event rate is likely due to the RCA amplicons of the preexisted let-7a in the extracted salivary RNAs rather than the salivary RNAs themselves. In fact, the gel analysis revealed that the size range of extracted salivary RNA is shorter than 500 nucleotides, shown in FIG. 7D. These smaller-sized background RNA is too small to be detected by our pores with 200 nm diameter, shown in FIG. 7C. The quantification experiments of miR-30e and miR-21 were also performed, as shown in FIGS. 9A-9F and 10A-10F.

### Specificity of RC-Coupled Nanopore Counting

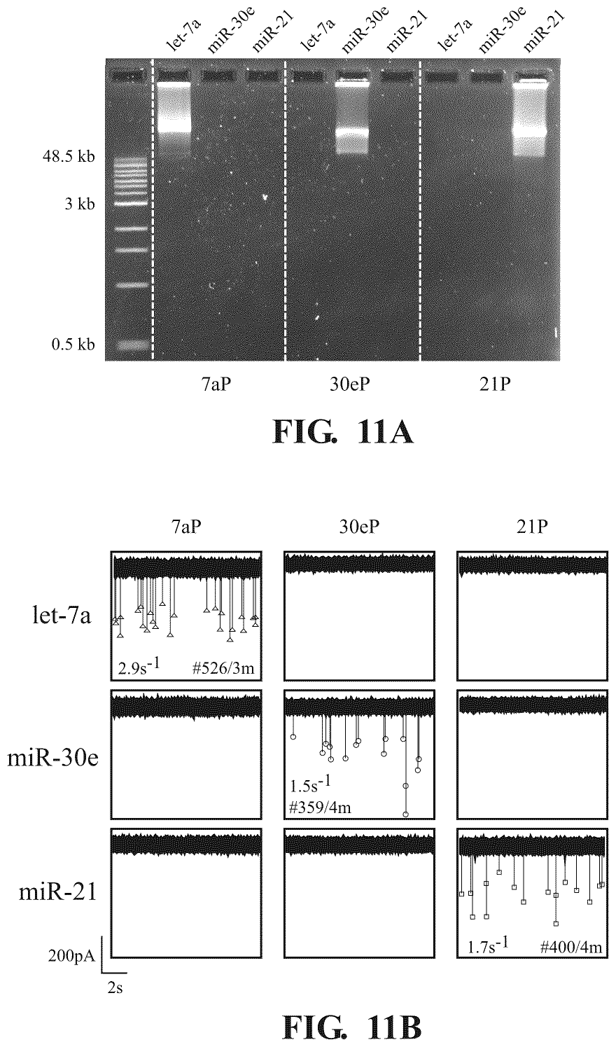

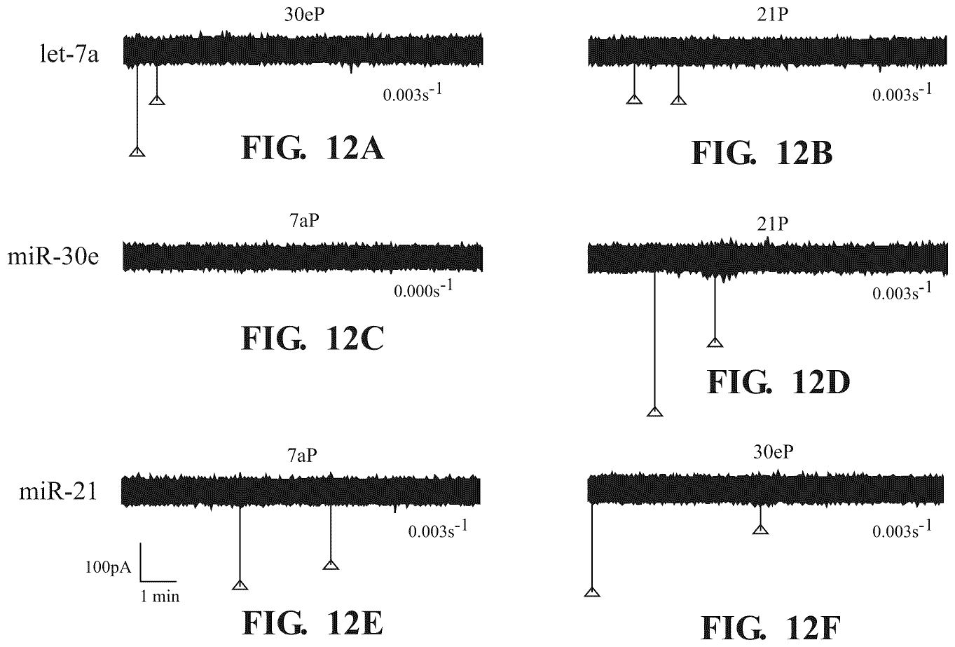

Due to the short length and high homogeneity of miRNAs, the specificity of designed padlock probes is vital for accurate miRNA identification and quantification^{11, 18}. To evaluate the specificity of our padlock probes against let-7a, miR-30e and miR-21, the cross-reactivity test was performed by running nine RCA reactions with different miRNA/probe combinations. The resulting amplicons were examined by the gel analysis, as shown in FIG. 11A. As shown, there were no bands observed for the non-specific combinations. Only the combinations of miRNA and its specific probe could produce the elongated RCA products with a size larger than 48.5 kb. These RCA products were subsequently analyzed by the glass sub-micron pore sensor. FIG. 11B plotted the representative current traces for each case (under 400 mV bias voltage). As expected, translocation events with a rate larger than 1 s⁻¹ were evident for the specific reactions, whereas the event rates were negligible for the non-specific reactions (<0.003 s⁻¹, see FIGS. 12A-12F). There is a significant event rate difference between the specific reaction and the non-specific reaction. This means the designed padlock probes are specific to their targets and there is no cross-reactivity among the panel members of let-7a, miR-30e and miR-21. In addition, the sub-micron pore sensor is only responsive to the specifically elongated ssDNAs without interference from the background molecules from RCA reactions.

Profiling mTBI-Related miRNAs from a Mixture

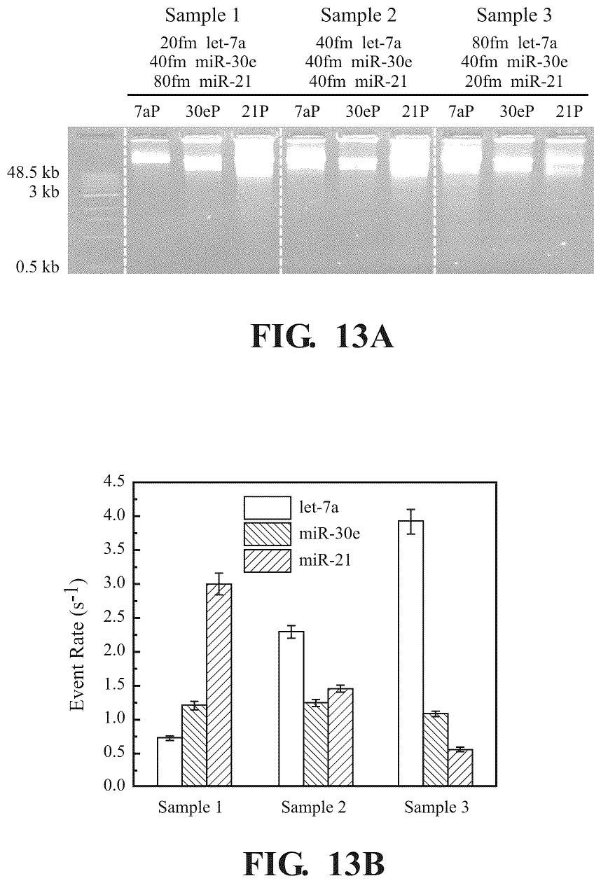

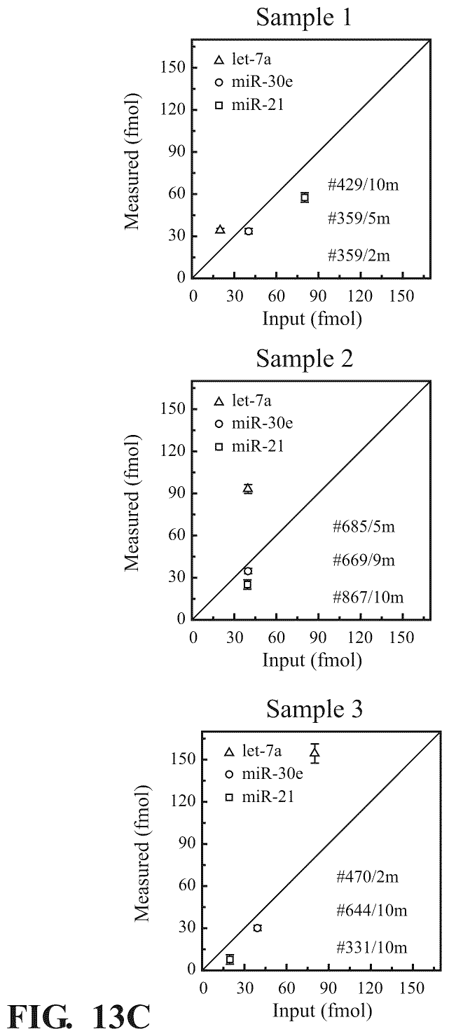

Recent studies have shown that a panel of multiple miRNAs represents a more accurate biomarker for mTBI^{7, 29 21, 22, 24, 30}. To evaluate the ability of the RCA-coupled nanopore counting platform to profile multiple types of miRNAs in a mixture, the quantification experiment was carried out using a mixture solution containing varying amounts of let-7a, miR-30e and miR-21. The relative abundance of each of these miRNAs was intentionally controlled. A total of three samples shown in FIG. 13A were tested. As shown in the gel images, there were clear RCA products for each of these mixture samples added into a specific probe, indicating the success of the RCA assay for the mixed samples.

The nanopore counting was then performed to quantify the miRNA constitutes. FIG. 13B plots the event rates for different miRNAs in each of these mixed samples. As can be seen, the event rates for miR-30e were consistent among these samples due to the same miRNA quantity (40 fmol). The relative event rates profile for let-7a and miR-21 from samples 1 to 3 qualitatively agrees with the input let-7a quantity in these samples. To test the quantitative agreement between the input and output, we used the correlation equation obtained in FIGS. 8C, 9C, and 10C to convert the event rate into the concentration. FIG. 13C presents the measured miRNA quantity versus the input miRNA quantity for three samples. A line with a slope of 1 was overlaid with the plot, representing an ideal measurement. As can be seen, while not all the data points fall on the ideal line, the measured quantity agrees very well with the input quantity. The relative abundance of let-7a, miR-30e and miR-21 in each of these mixed samples was correctly captured.

To understand the factors that lead to the measurement uncertainty, one can examine the event rate versus the analyte concentration relationship in nanopore counting. Previous work shows that the capture of 48.5 kbp DNA is diffusion-limited when using 10 nm glass nanopore²⁶. Since the glass nanopores used in our experiments are around 200 nm in diameter, it is large enough such that the transport is diffusion-limited rather than barrier-limited. It was known that the event rate can be linked to the analyte concentration C in the diffusion-limited region as R=2πμdΔVC³¹, in which μ is the free solution electrophoretic mobility, ΔV is the applied electric potential across the pore, and d is the characteristic length of the pore. The analyte (RCA amplicons) concentration C can be linked to the miRNA concertation C₀ as C=αC₀Tᵣ, in which α is the reaction efficiency and the Tᵣ is the reaction time. In our experiments, we used the same 0.4V bias voltage for all measurements; therefore, the ΔV would not contribute to the variations. In addition, the free solution electrophoretic mobility of DNA in the Tris-EDTA buffer was shown to be independent of the DNA length longer than 400 bp³², the contribution of the RCA product mobility to the event rate measurement can also be ruled out. Given the same reaction time Tᵣ, the measurement uncertainty is most likely due to the variations in nanopore characteristic length d and RCA reaction efficiency α. While all the nanopore devices we tested have a comparable aperture (217±9 nm), their actual geometry (characteristic length d) could be different. Therefore, the event rate counted by each device could be different. On the other hand, the RCA reaction efficiency a could vary between different miRNAs. This is consistent with previous observations that the hybridization³³, ligation³⁴ and elongation³⁵ efficiency could vary for different miRNAs and probe combinations. Although the event rate variations exist, they did show a good linear relationship (R²>97%) with input miRNA quantities when counting by a single nanopore device (FIG. 8F, 9F, and 10F).

As will be clear to those of skill in the art, the embodiments of the present invention illustrated and discussed herein may be altered in various ways without departing from the scope or teaching of the present invention. Also, elements and aspects of one embodiment may be combined with elements and aspects of another embodiment. It is the following claims, including all equivalents, which define the scope of the invention.

### REFERENCES

(1) Voss, J. D.; Connolly, J.; Schwab, K. A.; Scher, A. I. Update on the epidemiology of concussion/mild traumatic brain injury. Curr. Pain Headache Rep. 2015, 19 (7), 1-8.
(2) Davis, G. A.; Iverson, G.; Guskiewicz, K.; Ptito, A.; Johnston, K. Contributions of neuroimaging, balance testing, electrophysiology and blood markers to the assessment of sport-related concussion. Br. J. Sports Med. 2009, 43 (Suppl 1), i36-145.
(3) Gosselin, N.; Bottari, C.; Chen, J.-K.; Huntgeburth, S. C.; De Beaumont, L.; Petrides, M.; Cheung, B.; Ptito, A. Evaluating the cognitive consequences of mild traumatic brain injury and concussion by using electrophysiology. Neurosurg. Focus 2012, 33 (6), E7.
(4) Papa, L.; Ramia, M. M.; Kelly, J. M.; Burks, S. S.; Pawlowicz, A.; Berger, R. P. Systematic review of clinical research on biomarkers for pediatric traumatic brain injury. J. Neurotrauma 2013, 30 (5), 324-338.
(5) Bazarian, J. J.; Zemlan, F. P.; Mookerjee, S.; Stigbrand, T. Serum S-100B and cleaved-tau are poor predictors of long-term outcome after mild traumatic brain injury. Brain Inj. 2006, 20 (7), 759-765.
(6) Posti, J. P.; Hossain, I.; Takala, R. S.; Liedes, H.; Newcombe, V.; Outtrim, J.; Katila, A. J.; Frantzén, J.; Ala-Seppälä, H.; Coles, J. P. Glial fibrillary acidic protein and ubiquitin C-terminal hydrolase-LI are not specific biomarkers for mild CT-negative traumatic brain injury. J. Neurotrauma 2017, 34 (7), 1427-1438.
(7) Hicks, S. D.; Olympia, R. P.; Onks, C.; Kim, R. Y.; Zhen, K. J.; Fedorchak, G.; DeVita, S.; Rangnekar, A.; Heller, M.; Zwibel, H. Saliva microRNA biomarkers of cumulative concussion. Int. J. Mol. Sci. 2020, 21 (20), 7758.
(8) Ambros, V. The functions of animal microRNAs. Nature 2004, 431 (7006), 350-355.
(9) Leshkowitz, D.; Horn-Saban, S.; Parmet, Y.; Feldmesser, E. Differences in microRNA detection levels are technology and sequence dependent. RNA 2013, 19 (4), 527-538.
(10) Válóczi, A.; Hornyik, C.; Varga, N.; Burgyán, J.; Kauppinen, S.; Havelda, Z. Sensitive and specific detection of microRNAs by northern blot analysis using LNA-modified oligonucleotide probes. Nucleic Acids Res. 2004, 32 (22), e175-e175.
(11) Chen, C.; Ridzon, D. A.; Broomer, A. J.; Zhou, Z.; Lee, D. H.; Nguyen, J. T.; Barbisin, M.; Xu, N. L.; Mahuvakar, V. R.; Andersen, M. R. Real-time quantification of microRNAs by stem-loop RT-PCR. Nucleic Acids Res. 2005, 33 (20), e179-e179.
(12) Li, W.; Ruan, K. MicroRNA detection by microarray. Anal. Bioanal. Chem. 2009, 394 (4), 1117-1124.
(13) Weisz, H. A.; Kennedy, D.; Widen, S.; Spratt, H.; Sell, S. L.; Bailey, C.; Sheffield-Moore, M.; DeWitt, D. S.; Prough, D. S.; Levin, H. MicroRNA sequencing of rat hippocampus and human biofluids identifies acute, chronic, focal and diffuse traumatic brain injuries. Sci. Rep. 2020, 10 (1), 1-10.
(14) Koscianska, E.; Starega-Roslan, J.; Czubala, K.; Krzyzosiak, W. J. High-resolution northern blot for a reliable analysis of microRNAs and their precursors. Sci. World J. 2011, 11, 102-117.
(15) Hwang, D. W.; Song, I. C.; Lee, D. S.; Kim, S. Smart magnetic fluorescent nanoparticle imaging probes to monitor microRNAs. Small 2010, 6 (1), 81-88.
(16) Li, F.; Peng, J.; Wang, J.; Tang, H.; Tan, L.; Xie, Q.; Yao, S. Carbon nanotube-based label-free electrochemical biosensor for sensitive detection of miRNA-24. Biosens. Bioelectron. 2014, 54, 158-164.
(17) Deng, R.; Zhang, K.; Li, J. Isothermal amplification for microRNA detection: from the test tube to the cell. Acc. Chem. Res. 2017, 50 (4), 1059-1068.
(18) Cheng, Y.; Zhang, X.; Li, Z.; Jiao, X.; Wang, Y.; Zhang, Y. Highly sensitive determination of microRNA using target-primed and branched rolling-circle amplification. Angew. Chem. 2009, 121 (18), 3318-3322.
(19) Wanunu, M.; Dadosh, T.; Ray, V.; Jin, J.; McReynolds, L.; Drndić, M. Rapid electronic detection of probe-specific microRNAs using thin nanopore sensors. Nat. Nanotechnol. 2010, 5 (11), 807-814.
(20) Wang, Y.; Zheng, D.; Tan, Q.; Wang, M. X.; Gu, L.-Q. Nanopore-based detection of circulating microRNAs in lung cancer patients. Nat. Nanotechnol. 2011, 6 (10), 668-674.
(21) Svingos, A. M.; Asken, B. M.; Bauer, R. M.; DeKosky, S. T.; Hromas, G. A.; Jaffee, M. S.; Hayes, R. L.; Clugston, J. R. Exploratory study of sport-related concussion effects on peripheral micro-RNA expression. Brain Inj. 2019, 33 (4), 1-7.
(22) Hicks, S. D.; Johnson, J.; Carney, M. C.; Bramley, H.; Olympia, R. P.; Loeffert, A. C.; Thomas, N. J. Overlapping microRNA expression in saliva and cerebrospinal fluid accurately identifies pediatric traumatic brain injury. J. Neurotrauma 2018, 35 (1), 64-72.
(23) Harrison, E. B.; Hochfelder, C. G.; Lamberty, B. G.; Meays, B. M.; Morsey, B. M.; Kelso, M. L.; Fox, H. S.; Yelamanchili, S. V. Traumatic brain injury increases levels of miR-21 in extracellular vesicles: implications for neuroinflammation. FEBS Open Bio 2016, 6 (8), 835-846.
(24) Atif, H.; Hicks, S. D. A review of microRNA biomarkers in traumatic brain injury. J. Exp. Neurosci. 2019, 13, 1179069519832286.
(25) Blanco, L.; Bernad, A.; Lázaro, J. M.; Martin, G.; Garmendia, C.; Salas, M. Highly efficient DNA synthesis by the phage ϕ 29 DNA polymerase: Symmetrical mode of DNA replication. J. Biol. Chem. 1989, 264 (15), 8935-8940.
(26) Nouri, R.; Tang, Z.; Guan, W. Calibration-free nanopore digital counting of single molecules. Anal. Chem. 2019, 91 (17), 11178-11184.
(27) Meller, A.; Branton, D. Single molecule measurements of DNA transport through a nanopore. Electrophoresis 2002, 23 (16), 2583-2591.
(28) Tavallaie, R.; De Almeida, S. R.; Gooding, J. J. Toward biosensors for the detection of circulating microRNA as a cancer biomarker: an overview of the challenges and successes. Wiley Interdiscip. Rev.: Nanomed. Nanobiotechnol. 2015, 7 (4), 580-592.
(29) Di Pietro, V.; Porto, E.; Ragusa, M.; Barbagallo, C.; Davies, D.; Forcione, M.; Logan, A.; Di Pietro, C.; Purrello, M.; Grey, M. Salivary MicroRNAs: diagnostic markers of mild traumatic brain injury in contact-sport. Front. Mol. Neurosci. 2018, 11, 290.
(30) Di Pietro, V.; O'Halloran, P.; Watson, C. N.; Begum, G.; Acharjee, A.; Yakoub, K. M.; Bentley, C.; Davies, D. J.; Iliceto, P.; Candilera, G. Unique diagnostic signatures of concussion in the saliva of male athletes: the Study of Concussion in Rugby Union through MicroRNAs (SCRUM). Br. J. Sports Med. 2021, 1 (1), 103274. DOI: 10.1136/bjsports-2020-103274.
(31) Chen, P.; Gu, J.; Brandin, E.; Kim, Y.-R.; Wang, Q.; Branton, D. Probing single DNA molecule transport using fabricated nanopores. Nano Lett. 2004, 4 (11), 2293-2298.
(32) Stellwagen, E.; Stellwagen, N. C. Determining the electrophoretic mobility and translational diffusion coefficients of DNA molecules in free solution. Electrophoresis 2002, 23 (16), 2794-2803.
(33) Xu, S.; Zhan, J.; Man, B.; Jiang, S.; Yue, W.; Gao, S.; Guo, C.; Liu, H.; Li, Z.; Wang, J. Real-time reliable determination of binding kinetics of DNA hybridization using a multi-channel graphene biosensor. Nat. Commun. 2017, 8 (1), 1-10.
(34) Zhang, Z.; Lee, J. E.; Riemondy, K.; Anderson, E. M.; Yi, R. High-efficiency RNA cloning enables accurate quantification of miRNA expression by deep sequencing. Genome Biol. 2013, 14 (10), 1-13.
(35) Lee, S. Y.; Kim, K.-R.; Bang, D.; Bae, S. W.; Kim, H. J.; Ahn, D.-R. Biophysical and chemical handles to control the size of DNA nanoparticles produced by rolling circle amplification. Biomater. Sci. 2016, 4 (9), 1314-1317.

1. A method of counting target miRNAs, comprising the steps of:
providing padlock probes designed to specifically bind to the target miRNAs;
binding the miRNAs to corresponding probes forming a hybridized complex for each miRNA;
ligating the hybridized complex forming a closed circular structure;
elongating the hybridized complex using the probe as a template producing an elongated ssDNA product via rolling circle amplification (RCA) elongation;
providing a counting platform including a solid-state pore structure with a diameter greater than 10 nm;
measuring a concentration of the elongated ssDNA product through a translocation event rate through the pore structure of the counting platform; and
determining initial miRNA concentrations based on a quantity of the initial miRNA molecules, the quantity of the initial miRNA molecules being linear with the concentration of elongated ssDNA products.

2. The method of claim 1, wherein the diameter of the pore structure is smaller than 5 um.

3. The method of claim 1, wherein the diameter of the pore structure is 100-500 nm or 200-300 nm.

4. The method of claim 1, wherein the diameter of the pore structure is 250 nm.

5. The method of claim 1, wherein the elongated ssDNA product is greater than 70 k nucleotides.

6. The method of claim 1, wherein the pore structure is made from glass, Si, SiO2, SiNx, Zr02, HfO2, TiO2 (oxide dielectric material), 2D materials, or polymer materials.

7. The method of claim 1, wherein the miRNAs are salivary miRNAs related to mild traumatic brain injury (mTBI).

8. The method of claim 1, wherein the miRNAs are let-7a, miR-30e or miR-21.

9. The method of claim 1, wherein the target miRNAs include multiple miRNAs in a mixture.

10. A method of profiling multiple miRNA targets in an analyte mixture, comprising the steps of:
dividing the analyte mixture into a number of aliquots;
providing a padlock probe designed to specifically bind to one of the multiple miRNA targets for each aliquote;
binding each of the miRNA targets to its corresponding probe forming a hybridized complex for each miRNA target;
ligating each hybridized complex forming a closed circular structure;
elongating each hybridized complex using the respective probe as a template producing an elongated ssDNA product for each aliquote via rolling circle amplification (RCA) elongation;
providing a counting platform including a number of solid state pore structures each with a diameter greater than 10 nm;
providing one pore structure for one aliquote;
measuring a concentration of the elongated ssDNA product for each aliquote through a translocation event rate through the respective solid state pore structure in parallel; and
determining in parallel an initial concentration of each miRNA based on a quantity of the initial miRNA molecules, the quantity of the initial miRNA molecules being linear with the concentration of respective elongated ssDNA products.

11. The method of claim 10, wherein the diameter of each pore structure is smaller than 5 um.

12. The method of claim 10, wherein the diameter of each pore structure is 100-500 nm or 200-300 nm.

13. The method of claim 10, wherein the diameter of each pore structure is 250 nm.

14. The method of claim 10, wherein each elongated ssDNA product is greater than 70 k nucleotides.

15. The method of claim 10, wherein each pore structure is made from glass, Si, SiO2, SiNx, ZrO2, HfO2, TiO2 (oxide dielectric material), 2D materials, or polymer materials.

16. The method of claim 10, wherein the miRNAs are salivary miRNAs related to mild traumatic brain injury (mTBI).

17. The method of claim 10, wherein the miRNAs are let-7a, miR-30e or miR-21.
